(19) United States
(12) Patent Application Publication
(10) Pub. No.: US 2005/0112075 A1
(43) Pub. Date: May 26, 2005

(54) Reduction of hair growth

(76) Inventors: Cheng Shine Hwang, Framingham, MA (US); Gurpreet S. Ahluwalia, Wellesley, MA (US); Douglas Shander, Acton, MA (US)

Correspondence Address: FISH & RICHARDSON PC, 225 FRANKLIN ST, BOSTON, MA, 02110, US

(21) Appl. No.: 10/721,118

(22) Filed: Nov. 25, 2003

(51) Int. Cl. A61K 31/557; A61K 7/04; A61K 7/06

(52) U.S. Cl. 424/70.1; 514/573

(57) ABSTRACT

Mammalian hair growth is reduced by applying an agonist of prostaglandin DP-receptor.

### BACKGROUND

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, omithine decarboxylase, S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

There are four primary prostaglandins (PGs), namely, PGE₂, PGF₂, PGI₂ and PGD₂. They are synthesized and released from cells as a result of various chemical or physical stimuli. The released prostaglandins act as local hormones on cells in the vicinity to exert a variety of actions such as inflammation and muscle contraction or relaxation. Each prostaglandin binds to specific members of a family of G protein-coupled prostaglandin receptors. The interaction between prostaglandin and receptor activates intracellular G protein signal transduction pathways that alter the levels of second messengers such as cAMP, Ca²⁺ and inositol phosphates. It is this alteration of the second messenger levels that are thought to evokes a cell response. The receptor(s) for each prostaglandin is coupled to a unique signal transduction pathway. The specificity is such that aminor diversity in a prostaglandin's chemical structure can result in as much as complete opposite effect even in the same tissue. For example, the prostaglandin PGD₂ causes relaxation of smooth muscle, whereas PGE₂ induces the contraction of smooth muscle.

Chemically, prostaglandins contain a cyclopentane ring with the two-side chains alpha (−) and omaga (−). Prostaglandins are classified into prostaglandin types A through I based on modifications of the cyclopentane ring. However, the only naturally occurring prostaglandins are types D through I.

The cell receptors for the prostaglandins PGD₂, PGE₂, PGF₂ₐ, and PGI₂ are designated as DP, EP, FP and IP, respectively. The EP class of receptors is further divided into four subtypes: EP1, EP2, EP3, and EP4. Because of the diversity in prostaglandin receptors, as well as the complexity of prostaglandin signaling pathways, it is generally accepted that the knowledge about the function of any one of the prostaglandin in a given tissue cannot be extrapolated to another prostaglandin in the same tissue, or the same prostaglandin in another tissue. Each prostaglandin has a unique activity profile not exactly overlapping with others, indicating that each prostaglandin has a specific site of action.

| | G-protein-coupled |
|---|---|
| Prostaglandin | PG or Prostanoid |
| (PG) or Prostanoid | receptor* |
| PGE₂ | EP (EP1, EP2, EP3 and EP4) |
| PGF_{2α} | FP |
| PGI₂ | IP |
| PGD₂ | DP |

*The receptors are distinguished by their ligand-binding profiles, and the signal transduction pathways activated on ligand binding.

### SUMMARY

In one aspect, the invention provides a method (typically a cosmetic method) of reducing unwanted mammalian (preferably human) hair growth by applying to the skin an agonist of prostaglandin DP-receptor in an amount effective to reduce hair growth. Preferably, the agonist interacts strongly with the prostaglandin DP-receptor. The unwanted hair growth may be undesirable from a cosmetic standpoint or may result, for example, from a disease or an abnormal condition (e.g., hirsutism).

In another aspect, the invention provides a method of reducing unwanted mammalian hair growth by applying to the skin a compound selected from the group consisting of prostaglandin D₂, analogs or derivatives of prostaglandin D₂, PGJ₂, or an analog of PGJ₂.

In another aspect, the invention provides a method of reducing unwanted mammalian hair growth by applying to the skin a compound that activates DP receptor signal transduction pathway.

In a further aspect, the invention provides a method of reducing unwanted mammalian hair growth by applying to the skin a compound that inactivates prostaglandin D₂ metabolic pathway.

Typically, in practicing the aforementioned methods, the agonist/compound will be included in a topical composition along with a dermatologically or cosmetically acceptable vehicle. Accordingly, the present invention also relates to topical compositions comprising a dermatologically or cosmetically acceptable vehicle and an agonist of prostaglandin DP receptor. The present invention further relates to topical compositions comprising a dermatologically or cosmetically acceptable vehicle and (a) a compound selected from the group consisting of prostaglandin D₂, analogs or derivatives of prostaglandin D₂, PGJ₂, or an analog of PGJ₂; (b) a compound that activates DP receptor signal transmission pathway; and/or (c) a compound that inactivates prostaglandin D₂ metabolic pathway.

In addition, the present invention relates to the use of an agonist of prostaglandin DP-receptor for the manufacture of a therapeutic topical composition for reducing hair growth. Further, the present invention relates to the use of a compound for the manufacture of a therapeutic topical composition for reducing hair growth, wherein the compound is (a) a compound selected from the group consisting of prostaglandin D₂, analogs or derivatives of prostaglandin D₂, PGJ₂, or an analog of PGJ₂; (b) a compound that activates DP receptor signal transmission pathway; and/or or (c) a compound that inactivates prostaglandin D₂ metabolic pathway.

“Agonist of prostaglandin DP-receptor”, as used herein, means a compound that activates prostaglandin DP receptor. An agonist that “interacts strongly” with the prostaglandin DP-receptor is one that that binds the receptor with such affinity that it elicits a response that is at least approximately comparable to (in magnitude) to that elicited by prostaglandin D₂. “DP receptor”, as used herein, means the receptor is of the class that has the strongest affinity for PGD₂ of all the naturally occurring prostaglandins.

Specific compounds include both the compound itself and pharmacologically acceptable salts of the compound.

Other features and advantages of the invention may be apparent from the detailed description and from the claims.

### DETAILED DESCRIPTION

An example of a preferred composition includes at least one agonist of prostaglandin DP-receptor in a cosmetically and/or dermatologically acceptable vehicle. The composition may be a solid, semi-solid, or liquid. The composition may be, for example, a cosmetic and dermatologic product in the form of an, for example, ointment, lotion, foam, cream, gel, or solution. The composition may also be in the form of a shaving preparation or an aftershave. The vehicle itself can be inert or it can possess cosmetic, physiological and/or pharmaceutical benefits of its own.

Examples of agonists of prostaglandin DP-receptor include prostaglandin D₂ analogs and prostaglandin D₂-sequential metabolites and their analogs.

Analogs of prostaglandin D₂ (also referred to as PGD₂) are known. For example, U.S. Pat. No. 4,203,924 describes 2-decarboxy-2-hydroxymethyl-deoxy-9,10-didehydro-PGD₂ analogs; U.S. Pat. No. 4,201,873 describes 9-deoxy-9,10-didehydro-PGD₂ analogs; U.S. Pat. No. 4,562,204 describes trans-delta2-prostaglandin D₂ derivatives; U.S. Pat. No. 3,878,239 describes preparation of other prostaglandin D₂ analogs; and U.S. Pat. No. 5,700,835 describes 3-oxa-D-prostaglandins. Other examples of prostaglandin D₂ analogs are disclosed EP 0 098 141 and EP 0 097 023.

Prostaglandin D₂ sequential metabolites and their analogs also are known.

Specific examples of agonists are provided in Tables 1 and 1A.

TABLE I
Examples of PGD₂ analogs, PGD₂ sequential
metabolites and their analogs

| | |
|---|---|
| PGD₂ analog | 11-Deoxy-11-methylene PGD_{2,} |
| | 15(R)-15-methyl PGD_{2,} 15(S)-15-methyl |
| | PGD_{2,} 15-deoxy-Δ^{12,14}-PGD_{2,} 16, 16- |
| | dimethyl-PGD_{2,} 17-phenyl trinor |
| | PGD_{2,} 9β-halogen-15-cyclohexyl-prostaglandin, |
| | 11α-halogen-15-cyclohexyl-prostaglandin, |
| | ZK118182, RS93520, RS93427, SQ27986, |
| | ZK110841, BW245C, BW246C, BW A868C, |
| | L644122, and L644698. |
| PGD₂ metabolites | 13,14-Dihydro-15-keto PGD₂, |
| and PGJ₂ analogs | PGJ₂, Δ¹²-PGJ₂, |
| | 15-deoxy-Δ^{12,14}-PGJ_{2,} |
| | 9,10-dihydro-15-deoxy-Δ^{12,14}-PGJ₂ |

TABLE IA
Chemical names of the PGD₂ analogs from Table I

| | |
|---|---|
| ZK118182 | Acetic acid, [[(2Z)-4-[(1R,2R,3R,5R)- |
| | 5-chloro-2-[(1E,3S)-3-cyclohexyl-3- |
| | hydroxy-1-propenyl]-3-hydroxycyclopentyl]-2- |
| | butenyl]oxy]-(9CI) |
| RS93520 | Butanoic acid, 4-[(1R,2R,3S,6R)-2-[(3S)- |
| | 3-cyclohexyl-3-hydroxy-1-propynyl]-3- |
| | hydroxybicyclo[4.2.0]oct-7-ylidene]-, (4Z)-(9CI) |
| RS93427 | Butanoic acid, 4-[(1S,2S,3R,6S)-2-[(3S)-3- |
| | cyclohexyl-3-hydroxy-1-propynyl]-3- |
| | hydroxybicyclo[4.2.0]oct-7-ylidene]-, (4Z)-(9CI) |
| SQ27986 | 5-Heptenoic acid, 7-[(1S,2S,3S,4R)-3-[(1E,3S)- |
| | 3-cyclohexyl-3-hydroxy-1- |
| | propenyl]-7-oxabicyclo[2.2.1]hept-2-yl]-, (5Z)-(9CI) |
| ZK110841 | 5-Heptenoic acid, 7-[(1R,2R,3R,5R)- |
| | 5-chloro-2-[(1E,3S)-3-cyclohexyl-3- |
| | hydroxy-1-propenyl]-3-hydroxycyclopentyl]-, (5Z)-(9CI) |
| BW245C | 4-Imidazolidineheptanoic acid, 3-[(3R)-3- |
| | cyclohexyl-3-hydroxypropyl]-2,5- |
| | dioxo-, (4S)-rel-(9CI) |
| BW246C | (4R)-(3-[(3R,S)-3-Cyclohexyl-3-hydroxypropyl]- |
| | 2,5-dioxo)-4-imidazolidineheptanoic acid |
| L644122 | Benzoic acid, 4-[3-[3-[2-(1- |
| | hydroxycyclohexyl)ethyl]-4-oxo- |
| | 2-thiazolidinyl]propyl]-(9CI) |
| L644698 | Benzoic acid, 4-[3-[3-(3-hydroxyoctyl)-4- |
| | oxo-2-thiazolidinyl]propyl]-(9CI) |
| BWA868C | 4-Imidazolidineheptanoic acid, 3-[(2- |
| | cyclohexyl-2-hydroxyethyl)amino]-2,5- |
| | dioxo-1-(phenylmethyl)-(9CI) |

The composition may include more than one agonist of PGD₂. In addition, the composition may include one or more other types of hair growth reducing agents, such as those described in U.S. Pat. Nos. 4,885,289; 4,720,489; 5,132,293; 5,096,911; 5,095,007; 5,143,925; 5,328,686; 5,440,090; 5,364,885; 5,411,991; 5,648,394; 5,468,476; 5,475,763; 5,554,608; 5,674,477; 5,728,736; 5,652,273; WO 94/27586; WO 94/27563; and WO 98/03149, all of which are incorporated herein by reference.

The concentration of the agonist in the composition may be varied over a wide range up to a saturated solution, preferably from 0.1% to 30% by weight or even more; the reduction of hair growth increases as the amount of agonist applied increases per unit area of skin. The maximum amount effectively applied is limited only by the rate at which the agonist penetrates the skin. The effective amounts may range, for example, from 10 to 3000 micrograms or more per square centimeter of skin.

The vehicle can be inert or can possess cosmetic, physiological and/or pharmaceutical benefits of its own. Vehicles can be formulated with liquid or solid emollients, solvents, thickeners, humectants and/or powders. Emollients include stearyl alcohol, mink oil, cetyl alcohol, oleyl alcohol, isopropyl laurate, polyethylene glycol, petroleum jelly, palmitic acid, oleic acid, and myristyl myristate. Solvents include ethyl alcohol, isopropanol, acetone, diethylene glycol, ethylene glycol, dimethyl sulfoxide, and dimethyl formamide.

The composition optionally can include components that enhance the penetration of the agonist into the skin and/or to the site of action. Examples of penetration enhancers include urea, polyoxyethylene ethers (e.g., Brij-30 and Laureth-4), 3-hydroxy-3,7,11-trimethyl-1,6,10-dodecatriene, terpenes, cis-fatty acids (e.g., oleic acid, palmitoleic acid), acetone, laurocapram, dimethylsulfoxide, 2-pyrrolidone, oleyl alcohol, glyceryl-3-stearate, propan-2-ol, myristic acid isopropyl ester, cholesterol, and propylene glycol. A penetration enhancer can be added, for example, at concentrations of 0.1% to 20% or 0.5% to 5% by weight.

The composition also can be formulated to provide a reservoir within or on the surface of the skin to provide for a continual slow release of the agonist. The composition also may be formulated to evaporate slowly from the skin, allowing the agonist extra time to penetrate the skin.

A topical cream composition containing an agonist of PGD₂ may be prepared by mixing together water and all water soluble components in a mixing vessel-A. The pH is adjusted in a desired range from about 3.5 to 8.0. In order to achieve complete dissolution of ingredients the vessel temperature may be raised to up to 45° C. The selection of pH and temperature will depend on the stability of the agonist. The oil soluble components, except for the preservative and fragrance components, are mixed together in another container (B) and heated to up to 70° C. to melt and mix the components. The heated contents of vessel B are poured into the water phase (container A) with brisk stirring. Mixing is continued for about 20 minutes. The preservative components are added at temperature of about 40° C. Stirring is continued until the temperature reaches about 25° C. to yield a soft cream with a viscosity of 8,000-12,000 cps, or a desired viscosity. The fragrance components are added at about 25° C.-30° C. while the contents are still being mixed and the viscosity has not yet built up to the desired range. If it is desired to increase the viscosity of the resulting emulsion, shear can be applied using a conventional homogenizer, for example a Silverson L4R homogenizer with a square hole high sheer screen. The topical composition can be fabricated by including the agonist in the water phase during formulation preparation or can be added after the formulation (vehicle) preparation has been completed. The agonist can also be added during any step of the vehicle preparation. The components of cream formulations are described in the examples below.

### EXAMPLE # 1 (Cream):

| INCI Name | W/w (%) |
|---|---|
| DI Water | 61.00-75.00 |
| Agonist of PGD₂ | 1.00-15.00 |
| Mineral oil | 1.90 |
| Glyceryl stearate | 3.60 |
| PEG 100 stearate | 3.48 |
| Cetearyl alcohol | 2.59 |
| Ceteareth-20 | 2.13 |
| Dimethicone, 100 ct | 0.48 |
| Lipidure PMB^{a} | 3.00 |
| Advanced moisture complex^{b} | 5.00 |
| Stearyl alcohol | 1.42 |
| Preservative, fragrance | qs |
| and color pigment | |
| Total | 100.00 |

^{a}polyquartinium-51 (Collaborative Labs, NY);
^{b}glycerin and water and sodium PCA and urea and trehalose and polyqauternium-51 and sodium hyaluronate (Collaborative Labs, NY)

### EXAMPLE # 2 (Cream)

| INCI Name | w/w (%) |
|---|---|
| Agonist of PGD₂ | 0.5-15.00 |
| Glycerol (glycerin) | 0-5 |
| Isoceteth-20 | 3-7 |
| Glyceryl isostearate | 1.5-5 |
| Dicaprylyl ether | 3-15 |
| Glyceryl triacetate (triacetin) | 0.5-10 |
| Preservative, fragrance and color pigment | q.s. |
| Water | q.s. to 100.00 |

### EXAMPLE # 3 (Cream)

| INCI Name | w/w (%) |
|---|---|
| Agonist of PGD₂ | 0.5-15.00 |
| Glycerol (glycerin) | 0-5 |
| Isoceteth-20 | 3-7 |
| Glyceryl isostearate | 1.5-5 |
| Dicaprylyl ether | 3-15 |
| 1-dodecyl-2-pyrrolidanone | 0.5-10% |
| Preservative, fragrance and color | q.s. |
| Water | to 100.00 |

### EXAMPLE # 4 (Cream)

| INCI Name | w/w (%) |
|---|---|
| Water | 70 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| 1-Dodecyl-2-pyrrolidanone | 1-10 |
| Total | 100.00 |

An agonist of PGD₂ is added to the example 4 formulation and mixed until solubilized.

### EXAMPLE 5 (Cream)

| INCI Name | w/w (%) |
|---|---|
| Water | 70-80 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Monocaprylate/Caprate | 1-10 |
| (Estol 3601, Uniquema, NJ) | |
| Total | 100.00 |

An agonist of PGD₂ is added to the example 5 formulation and mixed until solubilized.

### EXAMPLE 6 (Cream)

| INCI Name | w/w (%) |
|---|---|
| Water | 70-80 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| cis Fatty acids | 1-10 |
| Total | 100.00 |

An agonist of PGD₂ is added to the example 6 formulation and mixed until solubilized.

### EXAMPLE 7(Cream)

| INCI Name | w/w (%) |
|---|---|
| Water | 70-80% |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Terpene(s) | 1-10 |
| Total | 100.00 |

An agonist of PGD₂ is added to the example 7 formulation and mixed until solubilized.

### EXAMPLE 8 (Cream)

| INCI Name | w/w (%) |
|---|---|
| Water | 70-80% |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Polyoxyethylene sorbitans (tween) | 1-10 |
| Total | 100.00 |

An agonist of PGD₂ is added to the example 8 formulation and mixed until solubilized.

A hydroalcoholic formulation containing an agonist of PGD₂ is prepared by mixing the formulation components in a mixing vessel. The pH of the formulation is adjusted to a desired value in the range of 3.5-8.0. The pH adjustment can also be made to cause complete dissolution of the formulation ingredients. In addition, heating can be applied to up to 45° C., or even up to 70° C. depending on the stability of the agonist to achieve dissolution of the formulation ingredients. The components of two hydroalcoholic formulations are listed below.

### EXAMPLE # 9 (Hydro-Alcoholic)

| INCI Name | w/w (%) |
|---|---|
| Water | 48.00-62.50 |
| An agonist of PGD₂ | 0.5-15.00 |
| Ethanol | 16.00 |
| Propylene glycol | 5.00 |
| Dipropylene glycol | 5.00 |
| Benzyl alcohol | 400 |
| Propylene carbonate | 2.00 |
| Captex-300^{a} | 5.00 |
| Total | 100.00 |

^{a}caprylic/capric triglyceride (Abitec Corp., OH).

### EXAMPLE # 10 (Hydro-Alcoholic)

| INCI Name | w/w (%) |
|---|---|
| Water | 53.00-67.9 |
| An agonist of PGD₂ | 0.1-15.00 |
| Ethanol | 16.00 |
| Propylene glycol | 5.00 |
| Dipropylene glycol dimethyl ether | 5.00 |
| Benzyl alcohol | 4.00 |
| Propylene carbonate | 2.00 |
| Total | 100.00 |

### EXAMPLE # 11 (HYDRO-ALCOHOLIC)

| INCI Name | w/w (%) |
|---|---|
| Ethanol (alcohol) | 80 |
| Water | 17.5 |
| Propylene glycol dipelargonate | 2.0 |
| Propylene glycol | 0.5 |
| Total | 100.00 |

An agonist of PGD₂ is added to the example 11 formulation and mixed until solubilized.

The composition should be applied topically to a selected area of the body from which it is desired to reduce hair growth. For example, the composition can be applied to the face, particularly to the beard area of the face, i.e., the cheek, neck, upper lip, and chin. The composition also may be used as an adjunct to other methods of hair removal including shaving, waxing, mechanical epilation, chemical depilation, electrolysis and laser-assisted hair removal.

The composition can also be applied to the legs, arms, torso or armpits. The composition is particularly suitable for reducing the growth of unwanted hair in women having hirsutism or other conditions. In humans, the composition should be applied once or twice a day, or even more frequently, to achieve a perceived reduction in hair growth. Perception of reduced hair growth could occur as early as 24 hours or 48 hours (for instance, between normal shaving intervals) following use or could take up to, for example, three months. Reduction in hair growth is demonstrated when, for example, the rate of hair growth is slowed, the need for removal is reduced, the subject perceives less hair on the treated site, or quantitatively, when the weight of hair removed (i.e., hair mass) is reduced.

Human hair Follicle Growth Assay:

Human hair follicles in growth phase (anagen) were isolated from face-lift tissue (obtained from plastic surgeons) under dissecting scope using a scalpel and watchmakers forceps. The skin was sliced into thin strips exposing 2-3 rows of follicles that could readily be dissected. Follicles were placed into 0.5 ml William's E medium (Life Technologies, Gaithersburg, Md.) supplemented with 2 mM L-glutamine, 10 μg/ml insulin, 10 ng/ml hydrocortisone, 100 units of penicillin, 0.1 mg/ml streptomycin and 0.25 μg/ml amphotericin B. The follicles were incubated in 24-well plates (1 follicle/well) at 37° C. in an atmosphere of 5% CO₂ and 95% air. Compounds are dissolved into dimethyl sulfoxide as 100-fold stock solution. The control hair follicles were treated with dimethyl sulfoxide without prostaglandin. The follicles were photographed in the 24-well plates under the dissecting scope at a power of 10×. Typically, image recordings were made on day 0 (day follicles were placed in culture), and again on day 7. The length of hair follicle was assessed using an image analysis software system. The growth of hair fiber was calculated by the subtracting the follicle length on day 0 from that determined on day 7.

PGD₂ and its analogs demonstrated a significant reduction of human hair follicle growth. All seven of the PGD₂ analogs tested significantly reduced hair growth. The results are provided in Table III.

TABLE III
Reduction of human hair follicle growth by PGD₂ and its analogs.

| | | Hair follicle length | | |
|---|---|---|---|---|
| | | increase (mm) | | |
| Prostaglandin | Dose (μM) | Treated | Control | % Reduction |
| PGD₂ | 30 | 0.24 ± 0.10 | 1.89 ± 0.38 | 87.3 ± 5.0 |
| 15-Deoxy-Δ^{12,14}-PGD₂ | 50 | 0.10 ± 0.08 | 1.76 ± 0.27 | 94.3 ± 4.5 |
| 16,16-Dimethyl PGD₂ | 50 | 0.10 ± 0.05 | 1.76 ± 0.27 | 94.3 ± 2.8 |
| 15(S)-15-Methyl PGD₂ | 100 | 0.08 ± 0.06 | 1.10 ± 0.35 | 92.7 ± 5.5 |
| 17-Phenyl trinor PGD₂ | 50 | 0.10 ± 0.12 | 1.62 ± 0.43 | 93.8 ± 7.4 |
| 11-Deoxy-11-methylene PGD₂ | 50 | 1.11 ± 0.14 | 1.76 ± 0.27 | 36.9 ± 8.0 |
| 15(R)-15-Methyl PGD₂ | 50 | 0.04 ± 0.05 | 1.62 ± 0.43 | 97.5 ± 3.1 |

The sequential metabolite of PGD₂, namely, PGJ₂ that is formed in the cells from PGD₂, was also found to reduce hair growth. In addition, natural metabolites and (analogs) of PGJ₂ that were tested reduced hair growth. The results are provided in Table IV.

TABLE IV
Reduction of human hair follicle growth by the PGD₂ metabolite,
PGJ₂ and its analogs.

| | | Hair follicle | | |
|---|---|---|---|---|
| | | length increase (mm) | | |
| Prostaglandin | Dose (μM) | Treated | Control | % Reduction |
| PGJ₂ | 30 | 0.16 ± 0.25 | 1.89 ± 0.38 | 91.5 ± 13 |
| Δ¹²-PGJ₂ | 30 | 0.29 ± 0.46 | 1.89 ± 0.38 | 84.6 ± 24 |
| 15-Deoxy-Δ^{12,14}- | 30 | 0.10 ± 0.09 | 1.89 ± 0.38 | 94.7 ± 5.0 |
| PGJ₂ | | | | |
| 9,10-Dihydro- | 50 | 0.02 ± 0.03 | 1.62 ± 0.43 | 98.8 ± 1.9 |
| 15-deoxy- | | | | |
| ^{Δ12,14}-PGJ₂ | | | | |

The hair growth inhibition profile by both PGD₂ and PGJ₂ (and its analogs) was found to be dose dependent. The results are provided in Table V.

TABLE V
Dose-dependent reduction of human hair follicle growth by
PGD₂, PGJ₂ and its metabolites.

| | | Growth of follicle (mm) | | |
|---|---|---|---|---|
| Prostaglandin | Dose (μM) | Treated | Control | % Reduction |
| PGD₂ | 1 | 1.51 ± 0.28 | 1.58 ± 0.30 | 4.4 |
| | 5 | 1.38 ± 0.30 | 1.58 ± 0.30 | 12.7 |
| | 10 | 0.98 ± 0.32 | 1.58 ± 0.30 | 38.0 |
| PGJ₂ | 1 | 1.43 ± 0.34 | 1.58 ± 0.30 | 9.5 |
| | 5 | 1.03 ± 0.35 | 1.58 ± 0.30 | 34.8 |
| | 10 | 0.50 ± 0.29 | 1.58 ± 0.30 | 68.4 |
| Δ-12-PGJ₂ | 1 | 1.40 ± 0.44 | 1.41 ± 0.34 | 0.7 |
| | 5 | 1.26 ± 0.38 | 1.41 ± 0.34 | 10.6 |
| | 10 | 0.32 ± 0.15 | 1.41 ± 0.34 | 77.3 |
| 15-deoxy-Δ^{12,14}- | 1 | 1.42 ± 0.37 | 1.41 ± 0.34 | 0 |
| PGJ₂ | 5 | 0.21 ± 0.13 | 1.41 ± 0.34 | 34.8 |
| | 10 | 0.08 ± 0.07 | 1.41 ± 0.34 | 94.3 |

Because PGD₂ and PGJ₂ analogs behave as agonists of the PGD₂, and are expected to bind strongly to this receptor, the results indicate that this stimulation of PGD₂ results in reduction of hair growth. These results further demonstrate that activation of PGD₂ by a compound that acts as an agonist of PGD₂ or PGJ₂ results in a reduction of hair growth.

Other embodiments are within the claims.

1. A method of reducing mammalian hair growth which comprises
selecting an area of skin from which reduced hair growth is desired; and
applying to said area of skin a dermatologically acceptable composition comprising an agonist of prostaglandin DP-receptor in an amount effective to reduce hair growth.

2. The method of claim 1, wherein said agonist is a prostaglandin D₂ analog.

3. The method of claim 1, wherein said agonist is a prostaglandin D₂ derivative.

4. The method of claim 1, wherein said agonist interacts strongly with the prostaglandin DP-receptor.

5. The method of claim 1, wherein said agonist is 11-deoxy-11-methylene PGD₂.

6. The method of claim 1, wherein said agonist is 15(R)-15-methyl PGD₂.

7. The method of claim 1, wherein said agonist is (S)-15-methyl PGD₂.

8. The method of claim 1, wherein said agonist is 15-deoxy-Δ^{12,14}-PGD₂.

9. The method of claim 1, wherein said agonist is 16,16-dimethyl-PGD₂.

10. The method of claim 1, wherein said agonist is 17-phenyl trinor PGD₂.

11. The method of claim 1, wherein said agonist is 9β-halogen-15-cyclohexyl-prostaglandin.

12. The method of claim 1, wherein said agonist is 11α-halogen-15-cyclohexyl-prostaglandin.

13. The method of claim 1, wherein said agonist is acetic acid, [[(2Z)-4-[(1R,2R,3R,5R)-5-chloro-2-[(1E,3 S)-3-cyclohexyl-3-L hydroxy-1-propenyl]-3-hydroxycyclopentyl]-2-butenyl]oxy]-(9CI).

14. The method of claim 1, wherein said agonist is butanoic acid, 4-[(1R,2R,3S,6R)-2-[(3S)-3-cyclohexyl-3-hydroxy-1-propynyl]-3-hydroxybicyclo[4.2.0]oct-7-ylidene]-, (4Z)-(9CI).

15. The method of claim 1, wherein said agonist is butanoic acid, 4-[(1S,2S,3R,6S)-2-[(3S)-3-cyclohexyl-3-hydroxy-1-propynyl]-3-hydroxybicyclo[4.2.0]oct-7-ylidene]-, (4Z)-(9CI).

16. The method of claim 1, wherein said agonist is 5-heptenoic acid, 7-[(1S,2S,3S,4R)-3-[(1E,3S)-3-cyclohexyl-3-hydroxy-1-propenyl]-7-oxabicyclo[2.2.1]hept-2-yl]-, (5Z)-(9CI).

17. The method of claim 1, wherein said agonist is 5-heptenoic acid, 7-[(1 R,2R,3R,5R)-5-chloro-2-[(1E,3 S)-3-cyclohexyl-3-hydroxy-1-propenyl]-3-hydroxycyclopentyl]-, (5Z)-(9CI).

18. The method of claim 1, wherein said agonist is 4-imidazolidineheptanoic acid, 3-[(3R)-3-cyclohexyl-3-hydroxypropyl]-2,5-dioxo-, (4S)-rel-(9CI).

19. The method of claim 1, wherein said agonist is (4R)-(3-[(3R,S)-3-cyclohexyl-3-hydroxypropyl]-2,5-dioxo)-4-imidazolidineheptanoic acid.

20. The method of claim 1, wherein said agonist is benzoic acid, 4-[3-[3-[2-(1-hydroxycyclohexyl)ethyl]-4-oxo-2-thiazolidinyl]propyl]-(9CI).

21. The method of claim 1, wherein said agonist is benzoic acid, 4-[3-[3-(3-hydroxyoctyl)-4-oxo-2-thiazolidinyl]propyl]-(9CI).

22. The method of claim 1, wherein said agonist is 4-imidazolidineheptanoic acid, 3-[(2-cyclohexyl-2-hydroxyethyl)amino]-2,5-dioxo-1-(phenylmethyl)-(9CI).

23. The method of claim 1, wherein said agonist is a PGD₂ metabolite.

24. The method of claim 1, wherein said agonist is 13, 14-dihydro-15-keto PGD₂.

25. The method of claim 1, wherein said agonist is PGJ₂.

26. The method of claim 1, wherein said agonist is Δ¹²-PGJ₂.

27. The method of claim 1, wherein said agonist is 15-deoxy-Δ^{12,14}-PGJ₂.

28. The method of claim 1, wherein said agonist is 9,10-dihydro-15-deoxy-Δ^{12,14} PGJ₂.

29. The method of claim 1, wherein the concentration of said agonist in said composition is between 0.1% and 30%.

30. The method of claim 1, wherein the composition provides a reduction in hair growth of at least 30% when tested in the Human Hair Follicle assay.

31. The method of claim 1, wherein the composition provides a reduction in hair growth of at least 60% when tested in the Human Hair Follicle assay.

32. The method of claim 1, wherein the agonist is applied to the skin in an amount of from 10 to 3000 micrograms of said agonist per square centimeter of skin.

33. The method of claim 1, wherein said mammal is a human.

34. The method of claim 33, wherein said area of skin is on the face of a human.

35. The method of claim 33, wherein the composition is applied to the area of skin in conjunction with shaving.

36. The method of claim 33, wherein said area of skin is on a leg of the human.

37. The method of claim 33, wherein said area of skin is on an arm of the human.

38. The method of claim 33, wherein said area of skin is in an armpit of the human.

39. The method of claim 33, wherein said area of skin is on the torso of the human.

40. The method of claim 1, wherein the composition is applied to an area of skin of a woman with hirsutism.

41. The method of claim 1, wherein said hair growth comprises androgen stimulated hair growth.

42. The method of claim 1, wherein the composition further includes a second component that also causes a reduction in hair growth.

43. A method of reducing mammalian hair growth, which comprises
selecting an area of skin including hair follicles from which reduced hair growth is desired; and
applying to the skin a compound selected from the group consisting of prostaglandin D₂, analogs of prostaglandin D₂, PGJ₂, or an analog of PGJ₂, in an amount effective to reduce hair growth.

44. A method of reducing mammalian hair growth, which comprises selecting an area of skin including hair follicles from which reduced hair growth is desired; and
applying to the skin a compound that activates DP receptor signal transduction pathway in an amount effective to reduce hair growth.

45. A method of reducing mammalian hair growth, which comprises
selecting an area of skin including hair follicles from which reduced hair growth is desired; and
applying to the skin a compound that inactivates prostaglandin D₂ metabolic pathway in an amount effective to reduce hair growth.
